SEQUENCE LISTING (7 sequences)

SEQ ID NO: 1 - DNA, 32 bp, Artificial Sequence
  Oligonucleotide
gctccgcgacggcagccacggcgtcggccggc

SEQ ID NO: 2 - DNA, 64 bp, Artificial Sequence
  Oligonucleotide
cgaggcgctgccgtcggtgccgcagccggccggtttctgctacgccggtaggctaacgttactg

SEQ ID NO: 3 - DNA, 32 bp, Artificial Sequence
  Oligonucleotide
gctccgcgacggcagccacggcgtcggccggg

SEQ ID NO: 4 - DNA, 32 bp, Artificial Sequence
  Oligonucleotide
gctccgcgacggcagccacggcgtcggccggt

SEQ ID NO: 5 - DNA, 32 bp, Artificial Sequence
  Oligonucleotide
gctccgcgacggcagccacggcgtcggcctgc

SEQ ID NO: 6 - DNA, 32 bp, Artificial Sequence
  Oligonucleotide
gctccgcgacggcagccacggcgtcggcctgg

SEQ ID NO: 7 - DNA, 32 bp, Artificial Sequence
  Oligonucleotide
gctccgcgacggcagccacggcgtcggcctgt

(19) United States
(12) Patent Application Publication
(10) Pub. No.: US 2003/0129607 A1
(43) Pub. Date: Jul. 10, 2003

(54) Compositions and methods for extension of nucleic acids

(75) Inventors: Thomas Kort, Germantown, MD (US); Mekbib Astatke, Germantown, MD (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 10/152,992

(22) Filed: May 23, 2002

Related U.S. Application Data
(60) Provisional application No. 60/293,182, filed on May 25, 2001.

(51) Int. Cl. C12Q 1/68; C07H 21/04; C12P 19/34

(52) U.S. Cl. 435/6; 435/91.2; 536/24.3

(57) ABSTRACT

The present invention relates to polypeptides, compositions and methods for extending nucleic acid molecules. Specifically, the present invention discloses polypeptides able to extend double-stranded and/or single-stranded nucleic acid molecules and/or single-stranded/double-stranded nucleic acid complexes (e.g., primer/template complexes, double-stranded templates, single-stranded templates or single-stranded primers) and more particularly when a mismatched dimer is present at or near the 3′ terminus of the primer. Accordingly, the polypeptide of the invention extends nucleic acids in a non-templated fashion. The invention also relates to kits for synthesizing and extending nucleic acid molecules comprising one or more of the polypeptides or compositions of the invention. The invention also relates to compositions prepared for carrying out the methods of the invention and to compositions made after or during such methods. The invention also generally relates to polypeptides and compositions useful for detecting and determining the site and characteristic of single polynucleotide polymorphisms.

### CROSS REFERENCE TO RELATED APPLICATIONS

This application claims priority to U.S. provisional patent application serial No. 60/293,182, filed May 25, 2001, which is specifically incorporated herein by reference in its entirety.

### BACKGROUND OF THE INVENTION

1. Field of the Invention

The genomes of viruses, bacteria, plants and animals naturally undergo spontaneous mutation in the course of their continuing evolution (Gusella, J. F., Ann. Rev. Biochem. 55:831-854 (1986)). Since such mutations are not immediately transmitted throughout all of the members of a species, the evolutionary process creates polymorphic alleles that co-exist in the species populations. In some instances, the mutation confers a survival or evolutionary advantage to the species. In some cases, such polymorphisms comprise mutations that are the determinative characteristic in a genetic disease. Indeed, such mutations may affect a single nucleotide in a protein-encoding gene in a manner sufficient to actually cause the disease (i.e., hemophilia, sickle-cell anemia, etc.). Despite the central importance of such polymorphisms in modem genetics, few methods have been developed that could permit the comparison of the alleles of two individuals at many such polymorphisms in parallel.

A “polymorphism” is a variation in the DNA sequence of some members of a species. A polymorphism is thus said to be “allelic,” in that, due to the existence of the polymorphism, some members of a species may have the predominant “unmutated” sequence (i.e. the original “allele”) whereas other members may have a less common “mutated” sequence (i.e. the variant or mutant “allele”). An allele may be referred to by the nucleotide(s) that comprise the mutation. Most polymorphisms arise from the replacement of only a single nucleotide from the predominant nucleic acid or gene sequence.

Several classes of polymorphisms have been identified. Variable number tandem repeats (“VNTRs”), for example arise from spontaneous tandem duplications of di- or trinucleotide or longer length repeated motifs of nucleotides (Weber, J. L., U.S. Pat. No. 5,075,217; Armour, J. A. L. et al., FEBS Lett. 307:113-115 (1992); Jones, L. et al., Eur. J. Haematol. 39:144-147 (1987); Horn, G. T. et al., PCT Application WO91/14003; Jeffreys, A. J., European Patent Application 370,719; Jeffreys, A. J., U.S. Pat. No. 5,175,082); Jeffreys, A. J. et al., Amer. J. Hum. Genet. 39:11-24 (1986); Jeffreys, A. J. et al., Nature 316:76-79 (1985); Gray, I. C. et al., Proc. R. Acad. Soc. Lond. 243:241-253 (1991); Moore, S. S. et al., Genomics 10:654-660 (1991); Jeffreys, A. J. et al., Anim. Genet. 18:1-15 (1987); Hillel, J. et al., Anim. Genet. 20:145-155 (1989); Hillel, J. et al., Genet. 124:783-789 (1990)). Variations which alter the lengths of the fragments that are generated by restriction endonuclease cleavage, the variations referred to as restriction fragment length polymorphisms (“RFLPs”). RFLPs have been widely used in human and animal genetic analyses (Glassberg, J., UK patent application 2135774; Skolnick, M. H. et al., Cytogen. Cell Genet. 32:58-67 (1982); Botstein, D. et al., Ann. J. Hum. Genet. 32:314-331 (1980); Fischer, S. G. et al. (PCT Application WO90/13668); Uhlen, M., PCT Application WO90/11369)).

“Single nucleotide polymorphism” or “SNPs” contains a polymorphic site, “X,” most preferably occupied by a single nucleotide, which is the site of the polymorphism's variation (Goelet, P. and Knapp, M., U.S. application Ser. No. 08/145,145, herein incorporated by reference). SNPs are more stable than other classes of polymorphisms. Their spontaneous mutation rate is approximately 10⁻⁹ (Kornberg, A., DNA Replication, W. H. Freeman & Co., San Francisco, 1980). SNPs occur at greater frequency, and with greater uniformity than RFLPs and VNTRs. Because SNPs result from sequence variation, new polymorphisms can be identified by sequencing random genomic DNA or cDNA molecules. Some RFLPs can also be considered a subset of SNPs because variation in the region of a RFLP can result in a single-base change in the region. SNPs can also result from deletions, point mutations and insertions. Any single base alteration, whatever the cause, can be a SNP. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., predisposition to cancer) reflects a mutation at a particular locus, then any polymorphism that is linked to the particular locus can be used to predict the probability that an individual will exhibit that trait.

SNPs can be characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or by other biochemical interpretation. However, no assay yet exists that is both highly accurate and easy to perform.

A. DNA Sequencing

The most obvious method of characterizing a polymorphism entails direct DNA sequencing of the genetic locus that flanks and includes the polymorphism. Such analysis is routinely accomplished using either the “dideoxy-mediated chain termination method,” also known as the “Sanger Method” (Sanger, F., et al., J. Molec. Biol. 94:441 (1975)) or the “chemical degradation method,” “also known as the “Maxam-Gilbert method” (Maxam, A. M., et al., Proc. Natl. Acad. Sci. (U.S.A.) 74:560 (1977)). Even though genomic sequence-specific amplification technologies, such as the polymerase chain reaction (Mullis, K. et al., Cold Spring Harbor Symp. Quant. Biol. 51:263-273 (1986); Erlich H. et al., European Patent Appln. 50,424; European Patent Appln. 84,796, European Patent Application 258,017, European Patent Appln. 237,362; Mullis, K., European Patent Appln. 201,184; Mullis, K. et al., U.S. Pat. No. 4,683,202; Erlich, H., U.S. Pat. No. 4,582,788; and Saiki, R. et al., U.S. Pat. No. 4,683,194)), may be employed to facilitate the recovery of the desired polynucleotides, these methods are technically demanding, relatively expensive, and have low throughput rates. As a result, there has been a demand for techniques that simplify repeated and parallel analysis of SNPs.

B. Exonuclease Resistance

Mundy, C. R. (U.S. Pat. No. 4,656,127) discusses alternative methods for determining the identity of the nucleotide present at a particular polymorphic site. Mundy's methods employ a specialized exonuclease-resistant nucleotide derivative. A primer complementary to the allelic sequence immediately 3′-to the polymorphic site is permitted to hybridize to a target molecule. If the polymorphic site on the target molecule contains a nucleotide that is complementary to the particular exonucleotide-resistant nucleotide derivative present, then that derivative will be incorporated by a polymerase onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. The Mundy method has the advantage that it does not require the determination of large amounts of extraneous sequence data. It has the disadvantages of destroying the amplified target sequences, and unmodified primer and of being extremely sensitive to the rate of polymerase incorporation of the specific exonuclease-resistant nucleotide being used.

C. Microsequencing Methods

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoll, L. et al., GATA 9:107-112 (1992); Nyren, P. et al., Anal. Biochem. 208:171-175 (1993)). These methods all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)). Such a range of locus-specific signals could be more complex to interpret, especially for heterozygotes. In addition, for some loci, incorporation of an incorrect deoxynucleotide can occur even in the presence of the correct dideoxynucleotide (Komher, J. S. et al., Nucl. Acids. Res. 17:7779-7784 (1989)). Such deoxynucleotide misincorporation events may be due to the kcat/Km of the DNA polymerase for the mispaired deoxy-substrate being comparable, in some sequence contexts, to the relatively poor kcat/Km of even a correctly base paired dideoxy-substrate (Komberg, A., et al., In: DNA Replication, Second Edition (1992), W. H. Freeman and Company, New York; Tabor, S. et al., Proc. Natl. Acad, Sci. (U.S.A.) 86:4076-4080 (1989)). This effect would contribute to the background noise in the polymorphic site interrogation.

D. Extension in Solution Using ddNTPs

Cohen, D. et al. (French Patent 2,650,840; PCT Appln. No.

WO91/02087) discuss a solution-based method for determining the identity of the nucleotide of a polymorphic site. As in the Mundy method of U.S. Pat. No. 4,656,127, a primer is employed that is complementary to allelic sequences immediately 3′-to a polymorphic site. The method determines the identity of the nucleotide of that site using labeled dideoxynucleotide derivatives, which, if complementary to the nucleotide of the polymorphic site will become incorporated onto the terminus of the primer.

The method of Cohen has the significant disadvantage of being a solution-based extension method that uses labeled dideoxynucleoside triphosphates. The target DNA template is usually prepared by a DNA amplification reaction, such as the PCR, that uses a high concentration of deoxynucleoside triphosphates, the natural substrates of DNA polymerases. These monomers will compete in the subsequent extension reaction with the dideoxynucleoside triphosphates. Therefore, following the PCR, an additional purification step is required to separate the DNA template from the unincorporated dNTPs. Because it is a solution-based method, the unincorporated dNTPs are difficult to remove and the method is not suited for high volume testing.

E. Solid-Phase Extension Using ddNTPs

An alternative method, known as Genetic Bit Analysis.TM. or GBA.TM. is described by Goelet, P. et al. (PCT Appln. No. 92/15712). In a preferred embodiment, the method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase. It is thus easier to perform, and more accurate than the method discussed by Cohen.

F. Oligonucleotide Ligation Assay

Another solid phase method that uses different enzymology is the “Oligonucleotide Ligation Assay” (“OLA”) (Landegren, U. et al., Science 241:1077-1080 (1988)). The OLA protocol uses two oligonucleotides which are designed to be capable of hybridizing to abutting sequences of a single strand of a target. One of the oligonucleotides is biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. OLA is capable of detecting point mutations. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990)). In this method, PCR is used to achieve the exponential amplification of target DNA, which is then detected using OLA. Assays, such as the OLA, require that each candidate dNTP of a polymorphism be separately examined, using a separate set of oligonucleotides for each dNTP. The major drawback of OLA is that ligation is not a highly discriminating process and non-specific signals can be a significant problem.

As will be appreciated, most of the above-described methods require a polymerase to discriminate incorporation of a paired nucleotide derivative onto the 3′-terminus of a primer molecule in a template directed fashion. It would be desirable to develop a more unique and selective process for discriminating single nucleotide polymorphisms. The present invention satisfies this need by providing a terminal nucleotidyl transferase, an enzyme that catalyzes, without template direction, the addition of one or more nucleotides (e.g., deoxynucleoside triphosphate) onto the 3′ hydroxy terminus of a nucleic acid. Thus, the invention is unique in that the mismatched nucleotide provides a positive signal, compared to previous techniques which detect mismatched nucleotide based on a negative result, for example, by the failure to extend a primer in a polymerase based assay. Unlike the polymerase-based assays, the method of the present invention enhances the extension of mispaired termini of primers as opposed to the extention of primers having termini complementary to the template of interest. Accordingly, the invention can be utilized to identify single nucleotide variations in genomic or of nucleic acid samples.

2. Related Art

The present invention relates to the field of molecular biology. More specifically, the invention is directed to methods and compositions for use in nucleic acid synthesis useful in detecting a single nucleotide variation present in a DNA strand and determining the identity of the nucleotide that is present at a particular site, such as a single nucleotide polymorphic (SNP) site, in the genomic DNA or RNA of an animal. The invention also relates to polypeptides and compositions which are capable of detecting such sites. The invention also relates to nucleic acid molecules encoding the polypeptides of the invention, and to vectors and host cells comprising such nucleic acid molecules. The invention also concerns kits comprising the compositions or polypeptides of the invention.

### BRIEF SUMMARY OF THE INVENTION

The present invention provides a method for nucleic acid synthesis by extending a mismatched dimer in a nucleic acid. A mismatched 3′-hydroxy terminus (e.g., one or a number of mismatched bases at a 3′ terminus) of the nucleic acid is preferentially recognized and extended by the method of the invention using any nucleoside triphosphate, altered or unaltered. In a preferred aspect, the invention is used to recognize single nucleotide mismatches or polymorphisms in double-stranded or single-stranded nucleic acid molecules.

More specifically, the invention relates to controlling nucleic acid synthesis by introducing a nucleotidyl transferase which is able to catalyze the addition of one or more nucleotides (e.g., at least one nucleoside triphosphate) to the 3′ hydroxy terminus of at least one nucleic acid. Substrate nucleic acid molecules for use in the invention include RNA or DNA or any modified or unmodified nucleic acid molecule. Nucleic acid molecules extended according to the invention are nucleic acid molecules having a single stranded portion (i.e., one or a number of bases that are not involved in base pairing) preferably at a 3′ terminus and include double stranded nucleic acid with at least one protruding 3′ hydroxy terminus, or double-stranded nucleic acid which may contain sequences which are not base paired with a complementary nucleic acid strand. Specifically included are double-stranded nucleic acid with at least one, including two, three, five, seven, ten, twelve, fifteen, twenty or even more nucleotides on a first strand that do not base pair with complementary bases on the second strand. In a preferred aspect, synthesis substrates (e.g., nucleic acids substantially extended by the peptides and compositions of the invention) are prepared by hybridization of at least one primer or oligonucleotide to at least one template (preferably forming a nucleic acid molecule, a portion of which is double stranded). Such a formed molecule preferably comprises at least one mismatched or unhybridized base at at least one 3′ terminus of the molecule. In a preferred aspect, the oligonucleotide or primer hybridizes to the template such that the 3′-most base of the oligonucleotide is aligned with a position known to be polymorphic in the template. In some embodiments, the template may be genomic DNA or an amplified fragment of a genomic DNA that contains a known polymorphic locus.

Nucleic acids that do not serve as synthesis substrate (e.g., not extended according to the invention) include double stranded molecules in which the 3′ termini of both strands are based paired. In some embodiments, the 3′-terminus of the oligonucleotide is base paired with a base at a known polymorphic locus in a template molecule. Non-synthesis substrate (e.g., nucleic acids substantially not extended by the invention) are preferably formed by hybridizing at least one primer or oligonucleotide to at least one template such that the 3′ terminus of the oligonucleotide and/or of the template are base paired. As is known in the art, a nuclease or a polymerase with nuclease activity can be used to cleave one strand to the point where 3′ termini are base paired and/or a polymerase can be used to extend one strand to the point where 3′ termini are base paired. Enzymes having significant 3′-5′-exonuclease activity are therefore not preferred in many embodiments of the invention. Such primers/oligonucleotides in the non-synthesis substrates preferably are completely complementary or fully hybridized to the template.

Nucleotidyl transferases of the invention preferably bind to or interact with nucleic acid molecules (e.g., nucleic acid synthesis substrates such as single stranded primers or single stranded templates or double-stranded molecules with a single stranded portion or portions) and continue nucleic acid synthesis in a non-templated fashion, for example by transferring at least one nucleotide at a mismatched base of the nucleic acid synthesis substrate.

The nucleotidyl transferases or peptides for use in the invention include enzymes or proteins which are members of family X DNA polymerases which lack homology to families A, B, and C. Members include the mammalian DNA polymerase b and terminal deoxynucleotidyl transferases. Family X DNA polymerases share a structurally similar (putative) active site. The X family of DNA polymerases has been reclassified based on structure and sequence comparison to be one of 7 known subfamilies of a nucleotidyltransferase superfamily (Holm and Sander, 1995, Trends Biochem Sci 20, 345-7). The subfamilies include 1) kanamycin nucleotidyl transferases, 2) polymerase family X which includes DNA polymerase b and TdT, 3) protein-P₁₁ uridyltransferase, 4) streptomycin 3′-adenyltransferase, 5) poly(A) polymerase, 6) (2′-5′) oligoadenylate synthetase, and 7) glutamine synthase adenyltransferase. Recently, the nucleotidyltransferase superfamily was further expanded based on the identification of a “minimal domain” using recently developed, sensitive computer methods (Aravind L. and E. Koonin, 1999, Nucleic Acids Res 27, 1609-18). The further expanded superfamily was named the Polβ superfamily. Additional members of the Polβ superfamily include 1) “minimal nucleotidyl-transferases, 2) TRF family of eukaryotic, chromatin associated nucleotidyl transferases, 3) putative signal transducing nucleotidyltransferases distantly related to GlnD and GlnE proteins, 4) proteobacterial adenylyl cyclase (a divergent member of the Polβ superfamily). Other polypeptides of the invention include NS5B, an RNA polymerase from hepatitis C virus (1996, EMBO J. 15, 12-22), and reverse transcriptases including those from AMV, MMLV, and HIV (Clark, J. M., 1988, Nucleic Acids Res. 16: 9677-9686; Paliska and Bendovic, 1992, Science 258: 1112-1118; Patel and Preston, 1994, Proc. Natl. Acad. Sci. USA 91: 549-553; Swanstrom, R. et al., 1981, J. Biol. Chem. 256: 1115-1121).

Thus, in a preferred aspect, at least one polypeptide having nucleotidyl transferase activity is introduced into the reaction mixture where it binds to or interacts with the substrate(s) (e.g., primer/template complexes, mismatched or complementary, double stranded molecules and/or single-stranded molecules such as single-stranded primers and single stranded templates) and, in the presence of at least one nucleotide desired to be added, extends the mismatched or unhybridized 3′-hydroxy terminus, preferably extending 3′ termini of the mismatched primers/templates at a higher efficiency compared to primers that are fully complementary with their template or that are base-paired at their 3′-terminus with their template. By mismatched primer is meant a primer containing at least one mismatch (i.e., a base that is not complementary to the base at the corresponding position in the template). The mismatched base may be at any position in the primer and a primer may contain more than one mismatched position. Mismatches are located in a primer preferably at at least one terminus (preferably at a 3′ terminus). Such a mismatch may result in a reduction of the melting temperature (Tm) of the primer/template pair as compared with a fully complementary primer/template. The ability of the polypeptides of the invention to interact with double-stranded nucleic acid substrates and/or single-stranded nucleic acid substrates and/or single-stranded/double-stranded complexes can be altered, i.e. the catalytic efficiency for complementary primer/templates can be improved as substrates by altering the reaction conditions, for example, by using buffers of low ionic strength or by changing divalent metal ions.

The invention therefore relates to a method for synthesizing one or more nucleic acid molecules, comprising (a) mixing one or more nucleic acid templates (which may be a DNA molecule such as genomic DNA, a fragment of a genomic DNA, a cDNA molecule, or an RNA molecule such as a mRNA molecule, or a template with modified nucleotides) with one or more primers, RNA, DNA, LNA (locked nucleic acid), PNA (peptide nucleic acid) or any primer with modified nucleotides such as 2′-OCH₃ modified nucleotide or phosphorothioate linkage, mismatched or complementary to the template, and one or more polypeptides or compositions of the present invention capable of binding or interacting with one or more double-stranded and/or single-stranded nucleic acid substrates and/or single-stranded/double-stranded complexes (e.g., substrates for nucleic acid synthesis such as templates, template/primer complexes and/or primers) wherein said polypeptide has the ability to catalyze, without template direction, the addition of one or more nucleoside triphosphates or modified nucleosides onto the 3′-hydroxy terminus of at least one nucleic acid, under conditions sufficient to extend the 3′-hydroxy terminus. Such incubation conditions may involve the use of one or more nucleotides or nucleosides and one or more nucleic acid synthesis buffers. The invention also relates to nucleic acid molecules synthesized or extended by this method.

More specifically, the invention relates to a method of extending a nucleic acid molecule comprising: (a) mixing a primer, wherein said primer is either complementary or mismatched to a sequence of the target molecule, and one or more polypeptides or compositions of the invention (e.g., a polypeptide with affinity to double-stranded nucleic acids and/or single-stranded nucleic acids and/or single-stranded/double-stranded complexes and having the ability to catalyze, without template direction, the addition of nucleoside triphosphate or modified nucleoside onto the 3′-hydroxyl terminus of a nucleic acid); (b) hybridizing said primer to said target molecule; (c) and incubating the mixture under conditions such that extension of the primer or target molecule by the polypeptide of the invention can occur. Preferably, the incubation conditions are accomplished at a temperature sufficient to activate the polypeptides of the invention. The invention also relates to nucleic acid molecules synthesized or extended by these methods.

In another aspect, the present invention relates to a method for detecting a single nucleotide polymorphism in a target nucleic acid molecule by providing one or more primers (exact match and/or mis-matched primers) one or more of which may be complementary at the 3′ end to an allelic sequence at the polymorphic site of the target molecule. A nucleoside triphosphate, which may be detectably labeled, is provided under conditions such that the 3′ hydroxyl terminus of the primer with a mismatch is elongated. If the polymorphic site on the target molecule contains a nucleotide that is not complementary to the allelic sequence, a mismatch will occur at the polymorphic site and the nucleoside triphosphate, which may be a labeled or otherwise modified nucleoside triphosphate, is added to the 3′ hydroxyl terminus of the mismatched primer, preferably at a rate or to an extent greater than the nucleotide is added to the 3′-terminus of an oligonucleotide that is base paired at the polymorphic site. The presence or absence of a single nucleotide variation can be detected, for example, by detecting the presence or absence of the labeled nucleotide or otherwise modified nucleoside triphosphate in the primer or by detecting a change in length of the primer, e.g., by gel electrophoresis. Preferably, the elongation is catalyzed by a nucleotidyl transferase.

In another aspect, the present invention provides a method for detecting the presence or absence of a nucleotide at a specific position in a template nucleic acid molecule by mixing a plurality of primers with the template and an enzyme (e.g., a nucleotidyl transferase) that extends mismatched primers with greater efficiency and/or at a greater rate than it extends primers that are exactly matched with the template. In some embodiments, four primers may be used. In some embodiments of this type, one primer may have a sequence that is complementary to the template (i.e., an exact match primer) while each of the other three primers may have a sequence that differs from the exact match primer by one or more nucleotides. In some embodiments, the sequence of each of the three primers may differ from the exact match primer and from each other by having a different nucleotide at one or more positions in each primer that correspond to the specific position in the template. For example, the template may have a T at the specific position to be detected, an exact match primer may contain an A at the position that would correspond to the specific position, while one of the other three primers may have a T at this same position, one the three primers may have a C at this same position and one of the three primers may have a G at this same position. In some embodiments, each of the four primers may be provided with a detectable label which may be the same or different. In some embodiments, each primer is provided with a different detectable label, for example, each primer may be provided with a fluorescent dye that has a different excitation and/or emission spectrum. The sequence of the primers may be selected such that the position in each primer that corresponds to the specific position in the template is at or near the 3′-end of the primer. Preferably, the sequence may be selected such that when the primers that do not exactly match the template are annealed to the template a complex is formed that has a single-stranded portion. The single-stranded portion thus formed may include the 3′-most nucleotide of the primer. To detect the presence of a specific nucleotide in the template, the primers are annealed to the template and contacted with the enzyme. The primers that do not exactly match the template at the specified position are preferentially extended compared to the exact match primer. The presence of extended primers is detected, for example, by gel electrophoresis and laser fluorescence. The primer that is least extended has the complementary base at the position in the primer corresponding to the specific position in the template.

The invention also relates to a positive method for detecting the presence of a single nucleotide variation in general; to the use of a positive detection method in conjunction with a negative detection method, each one serving to help confirm the results of the other; and to the use of a positive detection method that positively detects the single nucleotide variation and a second positive detection method that positively detects an absence of nucleotide variation.

The invention also relates to the polypeptides of the invention and to compositions comprising the polypeptides of the invention, to the primers (matched or mismatched) of the invention, to the target molecules or templates of the invention, to the extension products and nucleotide complementary to the primer, to the reaction conditions for extension reactions, as well as nucleic acid molecules encoding the polypeptides of the present invention, to vectors (which may be expression vectors) comprising these nucleic acid molecules, and to host cells comprising these nucleic acid molecules or vectors. The invention also relates to methods of producing a polypeptide encoded by nucleic acid molecules of the invention, comprising culturing the above-described host cells under conditions favoring the production of the polypeptide by the host cells, and isolating the polypeptide. The invention also relates to polypeptides produced by such methods.

The invention also relates to kits for use in synthesis of nucleic acid molecules, comprising one or more containers containing one or more of the polypeptides or compositions of the invention. These kits of the invention may optionally comprise one or more additional components selected from the group consisting of one or more nucleotides, one or more templates, one or more nucleotidyl transferases, one or more reverse transcriptases, one or more suitable buffers, one or more primers or modified primers, one or more terminating agents (such as one or more dideoxynucleotides or modified nucleotides such as 2′-OCH₃, ribose, LNA, etc.), and instructions for carrying out the methods of the invention. Suitable buffers are those buffers that permit interaction, e.g., enzymatic catalytic activity, of the components whose interaction is desired.

The invention also relates to compositions for use in synthesis of nucleic acid molecules or polypeptides and to compositions made for carrying out such synthesis reactions. The invention also relates to compositions made before, during or after carrying out the synthesis reactions of the invention. Such compositions of the invention may comprise one or more of the polypeptides of the invention and may further comprise one or more components selected from the group consisting of one or more nucleotides, one or more primers, one or more templates, one or more nucleotidyl transferases, one or more reverse transcriptases, one or more DNA polymerases, oen or more amino acid, one or more protein polymerase, one or more buffers, one or more buffer salts and one or more synthesized nucleic acid molecules made according to the methods of the invention.

Other preferred embodiments of the present invention will be apparent to one of ordinary skill in light of the following drawings and description of the invention, and of the claims.

### BRIEF DESCRIPTION OF THE DRAWINGS/FIGURES

FIG. 1 shows the relative extension of the 3′-termini of the primer strand catalyzed by terminal deoxynucleotide transferase (TdT). P denotes the position of the DNA primer (34-mer). Lanes a, b, c, d, e, and f denote the control oligonucleotide substrates (sequences as described in Examples below). Panels A, B, C, D, E and F indicate the elongation reaction using the respective oligonucleotide substrate by successive dGMP at 37° C. For each DNA substrate, the reaction was quenched at 5 and 30 minutes following the initiation of the reaction (started with the addition of TdT). The oligonucleotide substrate and the TdT concentrations were maintained at about 12.5 nM and 5 units/μl, respectively, for each reaction condition.

FIG. 2 shows the relative extension of the 3′-termini of the primer strand catalyzed by terminal transferase. P denotes the position of the DNA primer (34-mer). Lanes a, b and c denote the control oligonucleotide substrates (sequences are shown in Examples below). Panels A and B indicate the elongation reactions catalyzed by TdT of oligonucleotide substrates (see sequence II) by successive dGMP and dTMP, respectively, at 30° C. For each DNA substrate, the reaction was quenched at 2 and 10 minutes e.g., by sequestering Mg⁺⁺ with EDTA, following the initiation of the reaction (started with the addition of TdT). The oligonucleotide substrate and the TdT concentrations were maintained at about 10 nM and 5 units/μl, respectively, for each reaction condition.

### DETAILED DESCRIPTION OF THE INVENTION

Definitions

In the description that follows, a number of terms used in recombinant DNA technology are utilized extensively. In order to provide a clearer and consistent understanding of the specification and claims, including the scope to be given such terms, the following definitions are provided.

Primer. As used herein, “primer” refers to a single-stranded oligonucleotide that is extended by covalent bonding of nucleotide monomers during amplification or polymerization of a nucleic acid molecule. The primer can be DNA, RNA, PNA, LNA, phosphorothioate linkage and any modified nucleic acid. The primer can further be attached to a solid support. A mismatched primer as used herein refers to a primer as described above wherein the primer contains at least one mismatch at any position resulting in a reduction of the melting temperature (Tm) of the primer/template pair as compared with a fully complementary primer/template pair. Preferably the at least one mismatch is at at least one terminus, even more preferably at at least one 3′ terminus. The mismatch at a 3′ terminus is preferably a mismatch of the 3′ nucleotide, but may be a mismatch 2, 3, 4, 5, 6, 7 or more bases 5′ of the 3′ terminus. The mismatch may comprise 2, 3, 4, 5, 6, 7 or more bases that are mismatched.

Template. The term “template” as used herein with respect to applications other than terminal transferases refers to double-stranded or single-stranded nucleic acid molecules (RNA and/or DNA) which are to be amplified, synthesized or sequenced. In the case of a double-stranded molecule, denaturation of its strands to form a first and a second strand is preferably performed before these molecules are amplified, synthesized or sequenced, or the double-stranded molecule may be used directly as a template. For single stranded templates, a primer, mismatched or complementary to a portion of the template is hybridized under appropriate conditions and one or more polymerases or polypeptides of the present invention may then synthesize a nucleic acid molecule complementary to all or a portion of said template. Alternatively, for double-stranded templates, one or more promoters (e.g. SP6, T7 or T3 promoters) may be used in combination with one or more polymerases to make nucleic acid molecules complementary to all or a portion of the template. The newly synthesized (extended) molecules, according to the invention, may be equal to, longer than or shorter in length than the original template. Templates can be distinguished from primers by immobilization, labeling, size, sequence, blocking at the 3′ or 5′ end, and or any of the means to distinguish one nucleic acid from another that are known in the art.

Incorporating. The term “incorporating” as used herein means becoming a part of a DNA and/or RNA molecule or primer, or modified nucleic acid.

Amplification. As used herein “amplification” refers to any in vitro method for increasing the number of copies of a nucleotide sequence with the use of a polymerase cell free and cell culture methods are included. Nucleic acid amplification results in the incorporation of nucleotides into a DNA and/or RNA molecule or primer thereby forming a new molecule complementary to all or a portion of a template. The formed nucleic acid molecule and its template can be used as templates to synthesize additional nucleic acid molecules or can be used as templates of the invention. As used herein, one amplification reaction may consist of many rounds of replication. DNA amplification reactions include, for example, polymerase chain reactions (PCR). One PCR reaction may consist of, for example, 2, 3, 4 or 5 or more, even up to 100 or more “cycles” of denaturation and synthesis of a DNA molecule.

Nucleotide. As used herein “nucleotide” refers to a base-sugar-phosphate combination. Nucleotides are monomeric units of a nucleic acid sequence (e.g., DNA and RNA). The term nucleotide includes ribonucleoside triphosphates ATP, UTP, CTP, GTP and deoxyribonucleoside triphosphates such as dATP, dCTP, dITP, dUTP, dGTP, dTTP, or derivatives thereof. Such derivatives include, for example, [α-S]dATP, 7-deaza-dGTP, 7-deaza-dATP, and nucleotide derivatives that confer nuclease resistance or polymerase on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Oligonucleotide. “Oligonucleotide” refers to a synthetic or natural molecule comprising a covalently linked sequence of nucleotides which are joined by a phosphodiester or phosphorothioate (PNA) bond between the 3′ position of the deoxyribose or ribose of one nucleotide and the 5′ position of the deoxyribose or ribose of the adjacent nucleotide.

Protein. A poly amino acid “Protein” as used herein is interchangeable with “polypeptide.” Proteins can be as short as 2 amino acids, but preferably are as long at 5, 10, 15, 25, 40, 50, 80, 100, 125, 150, 200, 250, 300, 350, 400, 500, 600, 700, 800, 900, 1100 or more amino acids.

Hybridization. The terms “hybridization” and “hybridizing” refers to base pairing of two complementary single-stranded nucleic acid molecules (RNA and/or DNA, PNA, LNA) to give a double-stranded molecule. As used herein, two nucleic acid molecules may be hybridized, although the base pairing is not completely complementary. Accordingly, mismatched bases do not prevent hybridization of two nucleic acid molecules but reduce the melting temperature of said hybrids with respect to hybrids more completely complementary provided that appropriate conditions, well known in the art, are used.

Unit. The term “unit” as used herein refers to the activity of an enzyme. When referring, for example, to a DNA polymerase, one unit of activity is the amount of enzyme that will incorporate 10 nanomoles of dNTPs into acid-insoluble material (i.e., DNA or RNA) in 30 minutes under standard primed DNA synthesis conditions.

Vector. A plasmid, phagemid, cosmid or phage DNA or other DNA molecule which is able to replicate autonomously in a host cell, and which is characterized by one or a small number of recognition sites of one or more restriction endonuclease at which such DNA sequences may be cut in a determinable fashion without loss of an essential biological function of the vector, and into which DNA may be spliced in order to bring about its replication and cloning. The cloning vector may further contain or be made to contain a marker suitable for use in the identification of cells transformed with the cloning vector. Suitable markers, for example, are tetracycline resistance or ampicillin resistance. Many additional markers are known in the art.

Expression vector. A vector which is capable of enhancing the expression of a gene which has been cloned into it, after transformation into a host. A cloned gene is usually placed under the control of (i.e., operably linked to) certain control sequences such as promoter sequences.

Recombinant host. Any prokaryotic or eukaryotic microorganism or cells of higher organism which contains the desired cloned genes in an expression vector, cloning vector or any DNA molecule. The term “recombinant host” is also meant to include those host cells which have been genetically engineered to contain the desired gene in the host chromosome or genome.

Host. Any prokaryotic or eukaryotic microorganism or cells of a higher organism that is the recipient of a replicable expression vector, cloning vector or any DNA molecule. The vector cloning vector, or DNA molecule may contain, but is not limited to containing, a structural gene, a promoter and/or an origin of replication.

Promoter. A DNA sequence generally described as a part of the 5′ region of a gene, located proximal to the start codon. At the promoter region, transcription of an adjacent gene(s) under control of the promoter is initiated.

Gene. A nucleic acid sequence that contains information for making an RNA or DNA or expression of a polypeptide or protein.

Structural gene. A DNA sequence that is transcribed into messenger RNA that is then translated into a sequence of amino acids characteristic of a specific polypeptide.

Operably linked. As used herein means that the promoter is positioned to control the initiation of expression of the polypeptide encoded by the structural gene.

Expression. Expression is the process by which a gene produces a polypeptide. It includes transcription of the gene into messenger RNA (mRNA) and the translation of such mRNA into polypeptide(s).

Substantially Pure. As used herein “substantially pure” means that the desired purified protein or polypeptide is essentially free from undesired contaminating cellular contaminants which are associated with the desired protein or polypeptide in nature. Contaminating cellular components may include, but are not limited to, phosphatases, exonucleases, endonucleases or undesirable DNA polymerase enzymes.

Thermostable. As used herein “thermostable” refers to a polypeptide having polymerase activity (e.g., DNA polymerase and reverse transcriptase) which is resistant to inactivation by heat. By way of example, DNA polymerases synthesize the formation of a DNA molecule complementary to a single-stranded DNA template by extending a primer in the 5′ to 3′ direction. This activity for mesophilic DNA polymerases may be inactivated by heat treatment. For example, T5 DNA polymerase activity is totally inactivated by exposing the enzyme to a temperature of 90° C. for 30 seconds. As used herein, a thermostable polymerase activity is more resistant to heat inactivation than a mesophilic polymerase. However, a thermostable polymerase does not mean to refer to an enzyme which is totally resistant to heat inactivation and thus heat treatment may reduce the polymerase activity to some extent. A thermostable polymerase typically will also have a higher optimum temperature than mesophilic polymerases.

Solid support. Supports for use in accordance with the invention may be any support or matrix suitable for attaching nucleic acid molecules. Such molecules may be added or bound (covalently or non-covalently) to the supports of the invention by any technique or any combination of techniques known in the art. Supports of the invention may comprise, inter alia, nitrocellulose, diazocellulose, glass, polystyrene (including microtitre plates), polyvinylchloride, polypropylene, polyethylene, polyvinylidenedifluoride (PVDF), dextran, Sepharose, agar, starch and nylon. Supports of the invention may be in any form or configuration including beads, filters, membranes, sheets, fruits, plugs, columns and the like. Solid supports may also include multi-well tubes (such as microtitre plates) such as 12-well plates, 24-well plates, 48-well plates, 96-well plates, and 384-well plates, etc. Preferred beads are made of glass, latex or a magnetic material (magnetic, paramagnetic or superparamagnetic beads).

In a preferred aspect, methods of the invention may be used in preparation of arrays of proteins or nucleic acid molecules (RNA or DNA) or arrays of other molecules, compounds, and/or substances. Such arrays may be formed on microplates, glass slides or blotting membranes and may be referred to as microarrays or gene-chips depending on the format and design of the array. Uses for such arrays include gene discovery, gene expression profiling, genotyping (SNP analysis, pharmacogenomics, toxicogenetics) and the preparation of nanotechnology devices.

Synthesis and use of nucleic acid arrays and generally attachment of nucleic acids to supports have been described (see, e.g., U.S. Pat. Nos. 5,436,327, 5,800,992, 5,445,934, 5,763,170, 5,599,695 and 5,837,832). An automated process for attaching various reagents to positionally defined sites on a substrate is provided in Pirrung, et al. U.S. Pat. No. 5,143,854 and Barrett, et al. U.S. Pat. No. 5,252,743. For example, disulfide-modified oligonucleotides can be covalently attached to solid supports using disulfide bonds. (See Rogers et al., Anal. Biochem. 266:23-30 (1999).) Further, disulfide-modified oligonucleotides can be peptide nucleic acid (PNA) using solid-phase synthesis. (See Aldrian-Herrada et al., J. Pept. Sci. 4:266-281 (1998).) Thus, nucleic acid molecules can be added to one or more supports (including being added in arrays on such supports) and nucleic acids, proteins or other molecules and/or compounds can similarly be added to such supports (or arrays). Conjugation of nucleic acids to a molecule of interest are known in the art and thus one of ordinary skill can produce molecules for attachment to supports (in array format or otherwise) according to the invention.

Essentially, any conceivable support may be employed in the invention. The support may be biological, non-biological, organic, inorganic, or a combination of any of these, existing as particles, strands, precipitates or molecules that can be precipitated, gels, sheets, tubing, spheres, containers, capillaries, pads, slices, films, plates, slides, etc. The support may be amorphous or have any convenient shape, such as a disc, square, sphere, circle, etc. The support is preferably flat but may take on a variety of alternative surface configurations. For example, the support may contain raised or depressed regions which may be used to increase surface area, control exposure to physical or chemical agents or for synthesis or other reactions. The support and its surface preferably form a rigid support on which to carry out the reactions described herein. The support and its surface can also be chosen to provide appropriate light-absorbing characteristics. For instance, the support may be a polymerized Langmuir Blodgett film, functionalized glass, Si, Ge, GaAs, GaP, SiO₂, SiN₄, modified silicon, or any one of a wide variety of gels or polymers such as nylon, proteins, (poly)tetrafluoroethylene, (poly)vinylidenedifluoride, polystyrene, polycarbonate, or combinations thereof. Colorants or dyes may be added. Other support materials will be readily apparent to those of skill in the art upon review of this disclosure. In a preferred embodiment the support is flat glass or single-crystal silicon.

Thus, the invention includes methods for preparing arrays of nucleic acid molecules attached to supports. In some embodiments, one nucleic acid molecule will be attached directly to the support, or to a specific section of the support, and one or more additional nucleic acid molecules will be indirectly attached to the support via attachment to the nucleic acid molecule which is attached directly to the support. In such cases, the nucleic acid molecule which is attached directly to the support provides a site of nucleation around which a nucleic acid array may be constructed.

In one aspect, the invention provides supports containing nucleic acid molecules which are produced by methods of the invention. Complementary strands and/or expression products may also be produced from these bound nucleic acid molecules while the nucleic acid molecules remain bound to the support. Thus, compositions and methods of the invention can be used to qualify and/or identify nucleic acid or expression products and products produced by these expression products.

Further, nucleic acid molecules attached to supports may be released from these supports. Methods for releasing nucleic acid molecules include restriction digestion, recombination, and altering conditions (e.g., temperature, salt concentrations, etc.) to induce the dissociation of nucleic acid molecules from the support, e.g., nucleic acid molecules which have hybridized to more tightly bound nucleic acid molecules. Thus, methods of the invention include the use of supports to which nucleic acid molecules have been bound for the isolation of nucleic acid molecules.

Examples of compositions which can be formed by binding nucleic acid molecules to supports include “gene chips,” often referred to in the art as “DNA microarrays” or “genome chips” (see U.S. Pat. Nos. 5,412,087 and 5,889,165, and PCT Publication Nos. WO 97/02357, WO 97/43450, WO 98/20967, WO99/05574, WO 99/05591 and WO99/40105, the disclosures of which are incorporated by reference herein in their entireties). In various embodiments of the invention, these gene chips may contain two- and three-dimensional nucleic acid arrays described herein.

Terminal nucleotidyl transferases. Terminal nucleotidyl transferases are enzymes that catalyze, without template direction, the addition of deoxynucleoside or ribonucleoside triphosphate or modified nucleosides on the 3′-hydroxy terminus of DNA or RNA. Single-stranded DNA or double-stranded DNA with a protruding 3′ hydroxy-terminus is a preferred substrate. By protruding is meant a double stranded DNA with a 3′ overhang or a template/primer pair where one or more bases in the primer are mismatched resulting in a lower Tm and fraying of the 3′ end of the primer. Blunt ended double-stranded DNA or double-stranded DNA with a recessed 3′ hydroxyl terminus can serve as a template, albeit less efficiently. The catalytic efficiency can be improved for these substrates by using different reaction conditions.

The X family of DNA polymerases has been reclassified based on structure and sequence comparison to be one of 7 subfamilies of a nucleotidyl transferase superfamily (Holm L. and C. Sander, 1995, supra). The subfamilies include 1) kanamycin nucleotidyl transferases, 2) polymerase family X which includes DNA polymerase b and TdT, 3) protein-P₁₁ uridyltransferase, 4) streptomycin 3′-adenyltransferase, 5) poly(A) polymerase, 6) (2′-5′) oligoadenylate synthetase, and 7) glutamine synthase adenyltransferase.

Recently, the nucleotidyltransferase superfamily was further expanded based on the identification of a “minimal domain” using recently developed, sensitive computer methods (Aravind L. and E. Koonin, 1999, supra). The further expanded superfamily was named the Polβ superfamily. Additional members of the polβ superfamily include 1) “minimal” nucleotidyl transferases, 2) TRF family of eukaryotic, chromatin associated nucleotidyl transferases, 3) putative signal transducing nucleotidyl transferases distantly related to GlnD and GlnE proteins, 4) proteobacterial adenylyl cyclase (a divergent member of the Polβ superfamily). Other nucleotidyl transferases include NS5B, an RNA polymerase from hepatitis C virus (1996, EMBO J. 15: 12-22) and reverse transcriptases including AMV, MMLV and HIV (Clark, J. M., 1988, Nucleic Acids Res. 16, 9677-9686; Paliska and Bendovic, 1992, Science 258: 1112-1118; Patel and Preston, 1994, Proc. Natl. Acad. Sci. USA 91: 549-553; Swanstrom, R. et al., 1981, J. Biol. Chem. 256, 1115-1121).

Terminal doexynucleotidyl transferase (TdT) is found in prelymphocytes during times when recombinational events leading to immunoglobulin and T-cell receptor diversification is occuring. TdT is responsible for the template independent addition of nucleotides (N regions) on either side of the D region of V(D)J assemblies (Komori et al., 1993, Science 261, 1171-5). The junctions form the major hypervariable regions in the heavy chains of immunoglobulin molecules.

TdT amino acid sequences are broadly conserved across vertebrate species ranging from bony fish to mammals. Various proteolytic forms of TdT have been isolated from human (Peterson et al., 1984, Proc. Natl. Acad. Sci. USA 81, 4363-7), calf (Diebel et al, 1993, Adv. Exp. Med. Biol. 145, 37-60), pig (Kaneda et al., 1981, Adv. Exp. Med. Biol. 145, 13-18), murine (Boule et al, 1998, Mol. Biotechnol. 10, 199-208), chick (Penit et al., 1982, Adv. Exp. Med. Biol. 145, 61-73), frog (R. Brown, 1981, J. Biol. Chem. 256, 3627-9), fish (J. Hansen, 1997, Immunogenetics 46, 367-75), tobacco and wheat germ sources (Brodniewicz-Proba, 1980, Biochem. J. 191, 139-45). TDT has also been isolated in multiple forms from human leukemia cells (Deibel et al., 1982, Adv. Exp. Med. Biol. 145, 37-60). Any of the above TdT's or any portion, proteolytic or genetically altered, which can extend a 3′-hydroxy terminus, can be used in the methods of the invention.

Polypeptides of the present invention are preferably used in the present compositions and methods at a final concentration in a synthesis reaction sufficient to extend the 3′-hydroxy terminus of the nucleic acid. Polypeptides having terminal transferase activity are preferably used in the present methods at a final concentration in solution of about 0.01-0.1 units per microliter where a unit is defined as the amount of enzyme catalyzing incorporation of 1μ mol dATP into acid perceptible material in 1 hr and 37° C. in the activity assay conditions using d(A)₅₀ as a primer, about 0.1-5.0 units per microliter, about 5-50 units per microliter, about 50-100 units per microliter, about 100-500 units per microliter, about 500-1000 units per microliter, about 1000-5000 units per milliliter, or about 0.1-2000 units per microliter, and most preferably at a concentration of about 5 units per microliter. Various reaction conditions, e.g., conditions suggested by enzyme or polypeptide suppliers, are known in the art. Of course, other suitable conditions and concentrations of such polypeptides suitable for use in the invention will be apparent to one or ordinary skill in the art.

Other terms used in the fields of recombinant DNA technology and molecular and cell biology as used herein will be generally understood by one of ordinary skill in the applicable arts.

Methods of Nucleic Acid Synthesis

The polypeptides and compositions of the invention may be used in methods for the detection and/or synthesis of nucleic acids. In particular, it has been discovered that the present polypeptides and compositions are useful in extending mismatched primer/template nucleic acid. The present polypeptides and compositions may therefore be used in any method requiring the detection and/or synthesis of nucleic acid molecules, such as DNA (including cDNA) and RNA, or modified nucleic acids such as 2′-OCH₃ LNA, PNA, molecules. Methods in which the polypeptides or compositions of the invention may advantageously be used include, but are not limited to, nucleic acid synthesis methods, in particular in the detection of single nucleotide polymorphisms.

Nucleic acid detection and/or synthesis methods according to this aspect of the invention may comprise one or more steps. For example, the invention provides a method for extending mismatched primer/template nucleic acid comprising incubating the polypeptide of the present invention with a template and primer wherein one or more mismatches are present such that a 3′-hydroxyl terminus is protruding. A nucleoside triphosphate, preferably labeled, to be added to the 3′-hydroxyl terminus is provided under conditions such that addition of a nucleoside triphosphate to the 3′-hydroxyl terminus is achieved. According to this aspect of the invention, the nucleic acid templates may be DNA molecules such as genomic DNA, a cDNA molecule or library, or RNA molecules such as a mRNA molecule. Conditions sufficient to allow synthesis such as pH, temperature, ionic strength, metal ions, and incubation times are known in the art and may be optimized according to the skill of people in the art.

Furthermore, enzymes of the present invention for use in the invention may be obtained commercially, for example from Invitrogen, LT (Rockville, Md.), Perkin-Elmer (Branchburg, N.J.), New England BioLabs (Beverly, Mass.) or Boehringer Mannheim Biochemicals (Indianapolis, Ind.). Alternatively, polypeptides having terminal transferase activity may be isolated from their natural vertebrate or plant sources according to standard procedures for isolating and purifying natural proteins that are well-known to one of ordinary skill in the art (see, e.g., Houts, G. E., et al., J. Virol. 29:517 (1979)). In addition, such enzymes may be prepared by recombinant DNA techniques that are familiar to one of ordinary skill in the art (see, e.g., Kotewicz, M. L., et al., Nucl. Acids Res. 16:265 (1988); Soltis, D. A., and Skalka, A. M., Proc. Natl. Acad. Sci. USA 85:3372-3376 (1988)). Examples of enzymes having terminal transferase activity may be derived from the scientific literature and include inter alia all of those described in the present application.

In accordance with the invention, the input or template nucleic acid molecules or libraries may be prepared from populations of nucleic acid molecules obtained from natural sources, such as a variety of cells, tissues, organs or organisms. Cells that may be used as sources of nucleic acid molecules may be prokaryotic (bacterial cells, including those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces) or eukaryotic (including fungi (especially yeast's), plants, protozoans and other parasites, and animals including insects (particularly Drosophila cells), nematodes (particularly Caenorhabditis elegans cells), and mammals (particularly human cells)). Cells from natural sources include cells exposed in the environment or the laboratory to one or m ore physical or chemical agents. The agent(s) may cause mutations in the cells or exert selective pressure(s) on a cell population.

Once the starting cells, tissues, organs or other samples are obtained, nucleic acid molecules (such as DNA, RNA (e.g., mRNA or poly A+ RNA) molecules) may be isolated, or cDNA molecules or libraries prepared therefrom, by methods that are well-known in the art (See, e.g., Maniatis, T., et al., Cell 15:687-701 (1978); Okayama, H., and Berg, P., Mol. Cell. Biol. 2:161-170 (1982); Gubler, U., and Hoffman, B. J., Gene 25:263-269 (1983)).

In the practice of a preferred aspect of the invention, a first nucleic acid molecule may be synthesized by mixing a nucleic acid template obtained as described above, which is preferably a DNA molecule or an RNA molecule such as an mRNA molecule or a polyA+ RNA molecule, with one or more primers wherein the primers are selected such that a mismatch between the primer and template will be extended in the presence of the above-described polypeptides or compositions of the invention to form a mixture. Such extension is preferably accomplished in the presence of nucleotides (e.g., deoxyribonucleoside triphosphates (dNTPs), dideoxyribonucleoside triphosphates (ddNTPs) or derivatives thereof).

Of course, other techniques of nucleic acid synthesis in which the terminal transferase polypeptides, compositions and methods of the invention may be advantageously used will be readily apparent to one of ordinary skill in the art.

Following extension or synthesis by the methods of the present invention, the synthesized nucleic acid fragments may be isolated for further use or characterization. This step is usually accomplished by isolation or separation of the synthesized nucleic acid fragments by any physical or biochemical means including gel electrophoresis, capillary electrophoresis, chromatography (including, for example, sizing, affinity, ion, hydrophobicity and immunochromatography), density gradient centrifugation, immunoprecipitation and immunoadsorption. Separation of nucleic acid fragments by gel electrophoresis is particularly preferred, as it provides a rapid and highly reproducible means of sensitive separation of a multitude of nucleic acid fragments, and permits direct, simultaneous comparison of the fragments in several samples of nucleic acids. One can extend this approach, in another preferred embodiment, to isolate and characterize these fragments or any nucleic acid fragment amplified or synthesized by the methods of the invention. Thus, the invention is also directed to isolated nucleic acid molecules produced by the amplification or synthesis methods of the invention.

In this embodiment, one or more of the synthesized nucleic acid fragments are removed from the gel which was used for identification (see above), according to standard techniques such as capillary action, electroelution or physical excision. The isolated unique nucleic acid fragments may then be inserted into nucleotide vectors, including expression vectors, suitable for transfection or transformation of a variety of prokaryotic (bacterial) or eukaryotic (yeast, plant or animal including human and other mammalian) cells. Alternatively, nucleic acid molecules produced by the methods of the invention may be further characterized, for example by sequencing (i.e., determining the nucleotide sequence of the nucleic acid fragments), by methods described below and others that are standard in the art (see, e.g., U.S. Pat. Nos. 4,962,022 and 5,498,523, which are directed to methods of DNA sequencing).

In an embodiment of the present invention, a method for identifying single nucleotide polymorphisms (SNP) is described comprising isolating a nucleic acid sequence suspected of containing a SNP, providing one or more primers complementary and/or noncomplementary (mismatched) at the 3′ end to an allelic sequence at the polymorphic site of the target nucleic acid molecule. A labelled nucleotide triphosphate is provided under conditions such that the 3′ hydroxyl terminus of the primer with a mismatch is elongated preferentially over the complementary 3′ hydroxyl terminus. If the polymorphic site on the target molecule contains a nucleotide that is not complementary to the allelic sequence, a mismatch will occur at the polymorphic site and the labelled nucleotide triphosphate is preferentially added. The presence or absence of a single nucleotide variation can be determined by detecting presence or absence or relative abundance of the labelled nucleotide on the primer. The efficiency of elongation of the mismatch containing primer is higher than that for a matched primer. Preferably, the elongation is catalyzed by a terminal nucleotidyl transferase.

In another embodiment, the identity of the single nucleotide polymorphism can be determined using a method of the present invention. Four primers capable of hybridizing to a region suspected of containing a SNP are designed each having a different nucleotide at the 3′ end region, e.g. the primers vary with either A, T, C, or G. Preferably, the variation is at the 3′ terminus. The 3′ end of the primers is preferably at or close to the suspected SNP site. Each of the primers is labeled such that it can be identified from the other three primers, for example using different fluorescence labels. A nucleoside triphosphate is added along with a nucleotidyl transferase such that the 3′ hydroxyl terminus of the primer with a mismatch is elongated, preferably, the nucleoside triphosphate is modified for ease of identification and/or isolation. The mismatch can be a single base or may be more than one base either contiguous or not contiguous. The number of bases from the mismatch to the 3′ terminus can vary, e.g., a mismatch can be 1, 2, 3, 4, 5 or more bases from the 3′ terminus. Multiple mismatches or a mismatch several bases upstream from a 3′ terminus can enhance extension activity. Conditions, e.g., G-C content, temperature, salt content, etc. are known in the art for varying base pairing (melting). Conditions for optimizing detection of a mismatch at or near 3′ terminis for specific templates/primers can be determined by one of ordinary skill in the art without undue experimentation. Once the primer/template pair with the added nucleotide has/have been isolated, the SNP can be determined by identifying the primer sequence(s) which has been elongated. Alternatively, once the presence of a SNP is detected. The precise location and base at the SNP can be determined by sequencing the template species or by use of other primers. A set of primers can include primers with 3′ termini 5′ or 3′ of other 3′ termini within the set. Size or other markers can identify the primers elongated position on a chip can identify a SNP. Determination of the polymorphic sites of the present invention is useful in the context of a trait or disease for determining whether the presence or absence of a particular polymorphism correlates with a particular trait or disease and detection or identification of a SNP facilitates the construction of a genetic map of a target species. The identification of SNPs permits one to use complementary oligonucleotides as primers in PCR or other reactions to isolate and sequence novel nucleic acid sequences located on or on either side of the SNP. The invention includes such novel nucleic acid sequences. The genomic sequences isolated through the use of such primers can be transcribed into RNA which can be expressed as proteins. The present invention also includes such proteins, as well as antibodies and molecules capable of binding to such proteins.

Vectors and Host Cells

The present invention also relates to vectors which comprise a nucleic acid molecule encoding one or more of the polypeptides of the present invention. Further, the invention relates to host cells which contain the gene or genes encoding the polypeptides of the invention and preferably to host cells comprising recombinant vectors containing such gene or genes, and to methods for the production of the polypeptides of the invention using these vectors and host cells. Such host cells are preferably genetically engineered and used for production of recombinant polypeptides.

The vector used in the present invention may be, for example, a phage or a plasmid, and is preferably a plasmid. Preferred are vectors comprising cis-acting control regions to the nucleic acid encoding a polypeptide of interest. Appropriate trans-acting factors may be supplied by the host, supplied by a complementing vector or supplied by the vector itself upon introduction into the host.

In certain preferred embodiments in this regard, the vectors provide for specific expression of a polypeptide encoded by the nucleic acid molecules of the invention; such expression vectors may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids or bacteriophages, and vectors derived from combinations thereof, such as cosmids and phagemids.

The DNA insert should be operatively linked to an appropriate promoter, such as the phage lambda P_{L} promoter, the E. coli lac, trp and tac promoters. Other suitable promoters will be known to the skilled artisan. The gene fusion constructs may further contain sites for transcription initiation, termination and, in the transcribed region, a ribosome binding site for translation. The coding portion of the mature transcripts expressed by the constructs will preferably include a translation initiation codon positioned at the beginning, and a termination codon (UAA, UGA or UAG) appropriately positioned at the end, of the polynucleotide to be translated.

The expression vectors will preferably include at least one selectable marker. Such markers include, for example, tetracycline or ampicillin resistance genes useful for culturing E. coli and other select bacteria.

Exemplary vectors for use in the present invention include pQE70, pQE60 and pQE-9, available from Qiagen; pBS vectors, Phagescript vectors, Bluescript vectors, pNH8A, pNH16a, pNH18A, pNH46A, available from Stratagene; pcDNA3 available from Invitrogen; and pGEX, pTrxfus, pTrc99a, pET-5, pET-9, pKK223-3, pKK233-3, pDR540, pRIT5 available from Pharmacia. Other suitable vectors will be readily apparent to the skilled artisan.

Representative examples of appropriate host cells include, but are not limited to, baculovirus, yeast, native sources, and bacterial cells such as E. coli, Streptomyces spp., Erwinia spp., Klebsiella spp. and Salmonella typhimurium. Preferred as a host cell is E. coli, and particularly preferred are E. coli strains DH10B and Stb12, which are available commercially (Invitrogen Corp., Carlsbad, California). Plant and animal cells including fish, insect, mammal (including human) are also preferred. These cells may be cultured in vitro or in vivo. Cell and culture methods are known in the art.

Peptide Production

As noted above, the methods of the present invention are suitable for production of any polypeptide of any length, via insertion of the above-described nucleic acid molecules or vectors into a host cell and expression of the nucleotide sequence encoding the polypeptide of interest by the host cell. Introduction of the nucleic acid molecules or vectors into a host cell to produce a transformed host cell can be effected, for example, by calcium phosphate transfection, DEAE-dextran mediated transfection, cationic lipid-mediated transfection, electroporation, transduction, transformation of chemically competent cells, infection or other methods. Such methods are described in many standard laboratory manuals, such as Davis et al., Basic Methods In Molecular Biology (1986). Once transformed host cells have been obtained, the cells may be cultivated under any physiologically compatible conditions of pH and temperature, in any suitable nutrient medium containing assimilable sources of carbon, nitrogen and essential minerals that support host cell growth.

Recombinant polypeptide-producing cultivation conditions will vary according to the type of vector used to transform the host cells. For example, certain expression vectors comprise regulatory regions which require culturing at certain temperatures, or addition of certain chemicals or inducing agents to the cell growth medium, to initiate the gene expression resulting in the production of the recombinant polypeptide. Thus, the term “recombinant polypeptide-producing conditions,” as used herein, is not meant to be limited to any one set of cultivation conditions. Appropriate culture media and conditions for the host cells and vectors are well-known in the art.

Following production in the host cells, the polypeptide of interest may be isolated by one of many techniques known in the art. For example, to liberate the polypeptide of interest from the host cells, the cells may be permeabilized or lysed or ruptured. This lysis may be accomplished by contacting the cells with a hypotonic solution, by treatment with a cell wall-disrupting enzyme such as lysozyme, by sonication, by treatment with high pressure, or by a combination of the above methods. The polypeptide may also be secreted by the host cell if appropriate secretion signals are utilized. Other methods of bacterial cell disruption and lysis that are known to one of ordinary skill may also be used.

Following disruption or pemeabilization, the polypeptide may be separated from the cellular debris by any technique suitable for separation of components in complex mixtures. The polypeptide may then be purified by well known isolation techniques. Suitable techniques for purification include, but are not limited to, ammonium sulfate, PEG, or ethanol precipitation, acid extraction, electrophoresis, immunoadsorption, anion or cation exchange chromatography, phosphocellulose chromatography, hydrophobic interaction chromatography, affinity chromatography, immunoaffinity chromatography, size exclusion chromatography, liquid chromatography (LC), high performance LC (HPLC), fast performance LC (FPLC), hydroxylapatite chromatography and lectin chromatography.

Kits

The present invention also provides kits for use in the synthesis of a nucleic acid molecule and for use in detecting single nucleotide polymorphisms. Kits according to this aspect of the invention may comprise one or more containers, such as vials, tubes, ampules, bottles and the like, which may comprise one or more of the terminal transferases and/or compositions of the invention.

The kits of the invention may comprise one or more of the following components: (i) one or more polypeptides or compositions of the invention, (ii) one or more polymerases and/or reverse transcriptases, (iii) one or more suitable buffers, (iv) one or more nucleotides, and (v) one or more primers; (vi) one or more templates, (vii) one or more “gene chips”, and (viii) instructions for carrying out the methods of the invention.

Compositions

The present invention also relates to compositions prepared for carrying out the syntheses of the invention. Additionally, the invention relates to compositions made during or after carrying out methods of the invention. In a preferred aspect, a composition of the invention comprises one or more of the polypeptides of the invention. Such compositions may further comprise one or more components selected from the group consisting of: (i) one or more polymerases and/or reverse transcriptases, (ii) one or more suitable buffers, (iii) one or more nucleotides, (iv) one or more templates, (v) one or more primers, (vi) one or more templates/primer complexes, and (vii) one or more nucleic acid molecules made by the synthesis, amplification or sequencing methods of the invention.

The invention also relates to compositions comprising the polypeptides of the invention bound to or complexed with one or more nucleic acid molecules as well as the polypeptide(s)/nucleic acid molecule(s) complex found in such compositions or made during the methods of the invention.

It will be readily apparent to one of ordinary skill in the relevant arts that other suitable modifications and adaptations to the methods and applications described herein are apparent and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The qualitative nucleotidyl transferase activity of TdT (available from Invitrogen, LT) was determined using a double stranded DNA with template overhang substrates that contain 1) a complementary 3′-terminus on the primer strand and 2) a primer/template with a mismatch at a 3′ terminus. We used a G:G and G:T mismatched terminated substrates in order to determine the effect of the type of mismatch base-pair on the efficiency of extension of TdT. In addition, oligonucleotide substrates that contain an internal mismatch (T:C) were used in order to determine the effect of the single stranded portion at the 3′ terminus on the efficiency of the enzymatic activity. The sequences of the oligonucleotide substrates used in these assays are shown below.

Single mismatched substrates

| | |
|---|---|
| 1A) | SEQ ID NO:1 |

^{5′}GCT CCG CGA CGG CAG CCA CGG CGT CGG CCG GC^{3′}

SEQ ID NO:2

^{3′}CGA GGC GCT GCC GTC GGT GCC GCA GCC GGC CGG TTT
CTG CTA CGC CGG TAG GCT AAC GTT ACT G^{5′}

| | |
|---|---|
| 1B) | SEQ ID NO:3 |

^{5′}GCT CCG CGA CGG CAG CCA CGG CGT CGG CCG GG^{3′}
^{3′}CGA GGC GCT GCC GTC GGT GCC GCA GCC GGC CGG TTT
CTG CTA CGC CGG TAG GCT AAC GTT ACT G^{5′}

| | |
|---|---|
| 1C) | SEQ ID NO:4 |

^{5′}GCT CCG CGA CGG CAG CCA CGG CGT CGG CCG GT^{3′}
^{3′}CGA GGC GCT GCC GTC GGT GCC GCA GCC GGC CGG TTT
CTG CTA CGC CGG TAG GCT AAC GTT ACT^{5′}

Internal mismatch substrates (3′ nucleotide mis-
match terminus)

| | |
|---|---|
| 2A) | SEQ ID NO:5 |

^{5′}GCT CCG CGA CGG CAG CCA CGG CGT CGG CCT GC^{3′}
^{3′}CGA GGC GCT GCC GTC GGT GCC GCA GCC GGC CGG TTT
CTG CTA CGC CGG TAG GCT AAC GTT ACT^{5′}

| | |
|---|---|
| 2B) | SEQ ID NO:6 |

^{5′}GCT CCG CGA CGG CAG CCA CGG CGT CGG CCT GG^{3′}
^{3′}CGA GGC GCT GCC GTC GGT GCC GCA GCC GGC CGG TTT
CTG CTA CGC CGG TAG GCT AAC GTT ACT G^{5′}

| | |
|---|---|
| 2C) | SEQ ID NO:7 |

^{5′}GCT CCG CGA CGG CAG CCA CGG CGT CGG CCT GT^{3′}
^{3′}CGA GGC GCT GCC GTC GGT GCC GCA GCC GGC CGG TTT
CTG CTA CGC CGG TAG GCT AAC GTT ACT G^{5′}

### Example 1

The nucleic acid extension activity of TdT was determined using two sets of primer/template substrates in order to investigate the relative efficiency of extension between a fully annealed and mismatched 3′-terminus. The DNA substrates used for these assays were 34/60 mer primer/template. The 5′-terminus of the primer strand for each case was labeled with ³²P using T4 polynucleotide kinase.

The elongation reaction was initiated by the addition of TdT to a solution of the DNA substrate in the presence of dGTP and MgCl₂. The reaction concentration of the DNA was about 10 nM, dGTP was 750 μM and MgCl₂ was 1.5 mM. For the control reaction (lanes a, b, c, d, e, and f), TdT was not added. The reactions were stopped at 5 and 30 minutes following addition of TdT. The final reaction concentration of TdTwas 5 unit/μl.

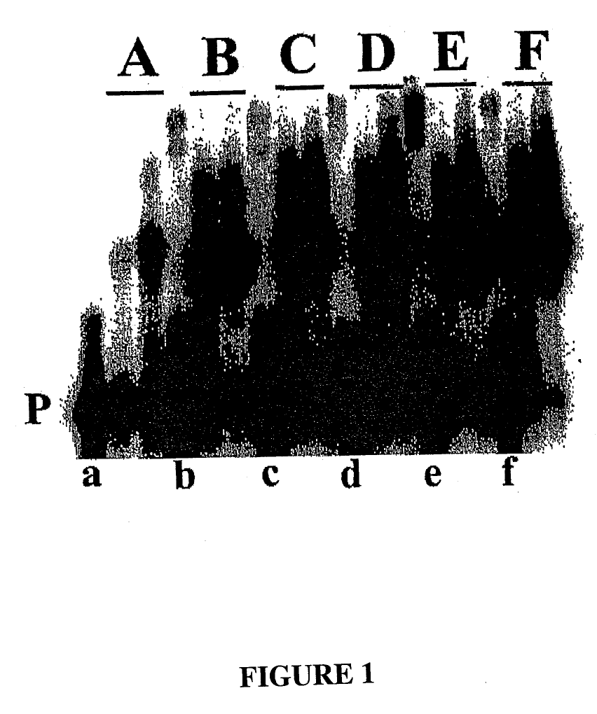

Results. As shown in FIG. 1, TdT extends primers that contain mismatches at the 3′-terminus with significantly enhanced efficiency compared to primers that are fully complementary with the template at the 3′-terminus. This result demonstrates that TdT can be utilized in order to identify mutations that are present in genomic templates. TdT extends G:G mis-paired termini with more efficiency compared to T:G termini. The addition of an internal mismatch at the third base from the 3′-terminus increased the rate of extension for each of the oligonucleotide sequences suggesting that the longer single stranded portion of the 3′-terminus is the preferred substrate for TdT.

### Example 2

The nucleic acid extension activity of TdT was determined using the rate of successive dGMP and dTMP insertions using DNA substrates. The relative efficiency of extension between a fully annealed and mismatched 3′-terminus by TdT using dGTP or dTTP was determined. The 5′-terminus of the primer strand was labeled with ⁼P using T4 polynucleotide kinase.

The elongation reaction was initiated by the addition of TdT to a solution of the DNA substrate in the presence of dGTP or dTTP and MgCl₂. The reaction concentration of the DNA was about 10 nM, dGTP/dTTP was 500 μM and MgCl₂ was 2 mM. For the control reaction (lanes a, b, and c), TdT was not added. The concentration of the TDT was 5 units/μl . The reactions were stopped at 2 and 10 minutes following addition of TDT.

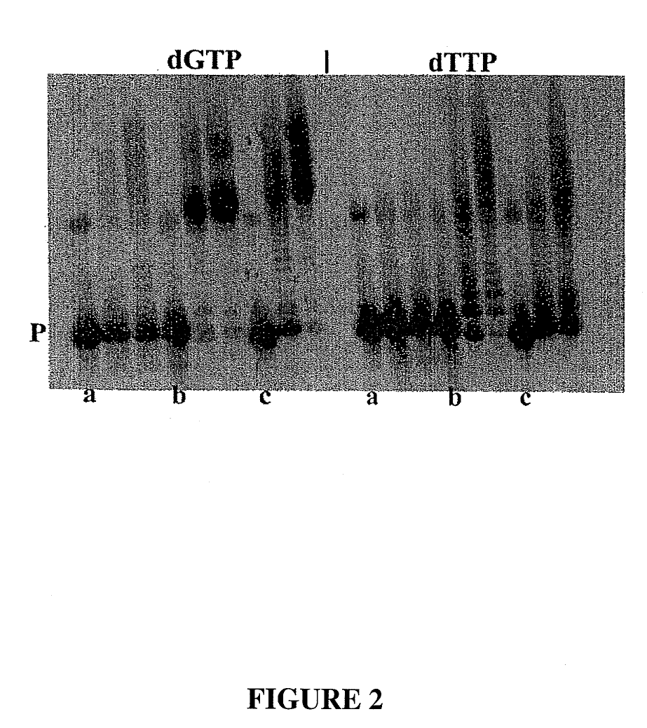

As shown in FIG. 2, TdT extends primers that contain mismatches at the 3′-terminus with significantly enhanced efficiency compared to primers that are fully complementary in each nucleotide triphosphate condition. Under our experimental condition, significant enhancement of extension is observed when the incoming is nucleotide is dGTP compared to dTTP.

Other polypeptides capable of preferentially extending mismatched primers are expected to function similarly to TdT for detecting/isolating mismatched, e.g., CNP containing, sequences.

Although the foregoing refers to particular embodiments, it will be understood that the present invention is not so limited. It will occur to those of ordinary skill in the art that various modifications may be made to the disclosed embodiments and that such modifications are intended to be within the scope of the present invention. All references, e.g., catalogues, patents, cited publications, mentioned herein are each incorporated by reference in their entireties.

What is claimed is:

1. A composition for primer extension comprising a polypeptide capable of extending a 3′-hydroxyl terminus of said primer wherein said polypeptide preferentially extends a mismatched primer over a fully complementary primer.

2. The composition of claim 1, wherein said polypeptide is selected from the polβ superfamily comprising subfamilies selected from the group consisting of: kanamycin nucleotidyl transferases, polymerase family X, protein P11 uridyltransferase, streptomycin 3′-adenyltransferase, poly(A) polymerase, 2′-5′ oligoadenylate synthetase, glutamine synthase adenyltransferase, “minimal” nucleotidyl-transferase, TRF family of eukaryotic, chromatin associated nucleotidyl transferases, putative signal transducing nucleotidyltransferases distantly related to GlnD and GlnE proteins, proteobacterial adenylyl cyclase, AMV reverse transcriptase, MMLV reverse transcriptase, HIV reverse transcriptase, and NS5B of hepatitis C virus.

3. A method for extending a primer molecule, comprising:
(a) mixing at least one nucleic acid primer with a template;
(b) adding one or more polypeptides of claim 1 to form a mixture; and
(c) incubating said mixture under conditions sufficient to extend said nucleic acid molecule at the 3′ hydroxyl terminus.

4. The method according to claim 3, wherein said synthesis is accomplished in the presence of at least one component selected from the group consisting of one or more nucleotides, and one or more primers.

5. The method according to claim 3, wherein said substrates are selected from the group consisting of a double-stranded nucleic acid template/primer complex, a single-stranded template/primer complex, and a single stranded/double stranded nucleic acid template/primer complex.

6. The method according to claim 3, wherein said nucleic acid is RNA.

7. The method according to claim 3, wherein said nucleic acid is DNA.

8. The method according to claim 3, wherein said nucleic acid is immobilized.

9. A nucleic acid molecule made according to the method of claim 3.

10. A method for identifying a single nucleotide polymorphism, SNP, in a target nucleic acid comprising:
(a) providing a primer complementary at least a portion of the 3′ end to an allelic sequence of the polymorphic site and a nucleotide transferase; and
(b) identifying the presence or absence of a SNP by detecting elongation or lack of elongation of said primer.

11. A method for identifying a single nucleotide polymorphism, SNP, in a target nucleic acid according to claim 10, comprising:
(a) providing a primer mismatched at the 3′ end to an allelic sequence of the polymorphic site and a nucleotide transferase; and
(b) identifying the presence or absence of a SNP by detecting the elongation of said primer.

12. The method according to claim 10 or 11, wherein said target nucleic acid is selected from the group consisting of a double-stranded molecule DNA, a single-stranded DNA, single stranded RNA, PNA, and LNA and mixtures thereof.

13. The method according to claim 10 or 11 wherein said target nucleic acid is immobilized.

14. The method according to claim 10 or 11 wherein said primer is immobilized.

15. The method according to claim 10 or 11 wherein the conditions in (b) present for said identifying include providing a terminal nucleotide transferase.

16. The method according to claim 15 wherein said terminal nucleotidyl transferase is chosen from the group consisting of the polβ superfamily comprising subfamilies selected from the group consisting of: kanamycin nucleotidyl transferases, polymerase family X, protein P11 uridyltransferase, streptomycin 3′-adenyltransferase, poly(A) polymerase, 2′-5′ oligoadenylate synthetase, glutamine synthase adenyltransferase, “minimal” nucleotidyl-transferase, TRF family of eukaryotic, chromatin associated nucleotidyl transferases, putative signal transducing nucleotidyltransferases distantly related to GlnD and GlnE proteins, proteobacterial adenylyl cyclase, AMV reverse transcriptase, MMLV reverse transcriptase, HIV reverse transcriptase, and NS5B of hepatitis C virus.

17. A kit for use in extending a nucleic acid molecule, said kit comprising one or more of the polypeptides of claim 1.

18. The kit of claim 17, further comprising one or more components selected from the group consisting of one or more nucleotides, one or more suitable buffers, and one or more primers.

19. A kit for use in detecting single nucleotide polymorphisms, said kit comprising one or more of the polypeptides of claim 1.

20. The kit of claim 19, further comprising one or more components selected from the group consisting of one or more nucleotides, one or more suitable buffers, and one or more primers.

21. An isolated and purified terminal transferase.

22. A vector comprising a gene encoding the transferase of claim 21.

23. A host cell comprising the vector of claim 22.

24. A host cell comprising a gene encoding the transferase of claim 21.

25. A method of producing a polypeptide, said method comprising:
(a) culturing the host cell of claim 23;
(b) expressing said gene; and
(c) isolating said polypeptide from said host cell.

26. A method of extending a nucleic acid molecule comprising:
(a) mixing at least one nucleic acid template with one or more polypeptides of claim 21; and
(b) incubating said mixture under conditions sufficient to allow extension of said nucleic acid molecule.

27. The composition of claim 1, wherein said polypeptide is bound to one or more nucleic acid molecules.

28. A method for detecting a single nucleotide polymorphism (SNP) wherein a first positive result is indicative of the presence of said SNP.

29. The method of claim 28, wherein the positive result is a nucleic acid having a 3′ extension region.

30. The method of claim 28, wherein a second positive result is indicative of the absence of said SNP.

31. The method of claim 29, wherein the 3′ extension region is 3′ of at least one mismatched base.

32. The method of claim 28, wherein the first positive result is a product of a nucleotidyl transferase.

33. The method of claim 30, wherein the second positive result is a nucleic acid having a 3′ extension region.

34. The method of claim 33, wherein the second positive result is an extension product whose sequence is determined by a nucleic acid template.

35. A method for detecting a nucleotide mismatch wherein a first positive result is indicative of the presence of said nucleotide mismatch.

36. The method of claim 35, wherein the positive result is a nucleic acid having a 3′ extension region.

37. The method of claim 35, wherein a second positive result is indicative of the absence of said nucleotide mismatch.

38. The method of claim 36, wherein the 3′ extension region is 3′ of at least one mismatched base.

39. The method of claim 35, wherein the first positive result is a product of a nucleotidyl transferase.

40. The method of claim 37, wherein the second positive result is a nucleic acid having a 3′ extension region.

41. The method of claim 40, wherein the second positive result is an extension product whose sequence is determined by a nucleic acid template.

42. The composition of claim 1, further comprising with said primer a mismatched or matched template with which said primer hybridizes.

43. The composition of claim 42, further comprising a solid support for attachment of said primer or said template.

44. The composition of claim 43, wherein said primer or said template is attached to said solid support.

45. The method of claim 42, comprising at least 2 primers, at least one of which is mismatched with respect to said template.

46. The method of claim 42, wherein said primer is mismatched at at least 2 nucleotides with respect to said template.

47. The method according to claim 3, wherein at least one of the one or more polypeptides is immobilized.

48. The method according to claim 10 or 11, wherein the nucleotidyl transferase is immobilized.

49. The composition of claim 27, wherein said one or more nucleic acid molecules comprises said primer.

50. The composition of claim 49, wherein said primer is bound as a template, said primer and said template being mismatched at at least one nucleotide at a 3′ end.

51. A composition comprising a nucleic acid template molecule, a plurality of different primers, and a nucleotidyl transferase enzyme.

52. A composition according to claim 51, comprising four different primers.

53. A composition according to claim 52, wherein each primer has a sequence that differs from the sequence of the other primers by one nucleotide at the same position in each primer.

54. A composition according to claim 53, wherein the position at which the sequences of the primers differ is the 3′-most nucleotide.

55. A composition according to claim 53, wherein the position at which the sequences of the primers differ is within 5 nucleotides of the 3′-terminus of the primers.
